(19) United States
(12) Patent Application Publication
(10) Pub. No.: US 2008/0004475 A1
(43) Pub. Date: Jan. 3, 2008

(54) PROCESS FOR THE PRODUCTION OF NEOPENTYLGLYCOL USING FORMALDEHYDE WITH A LOW METHANOL CONTENT

(75) Inventors: Tilman Sirch, Schifferstadt (DE); Michael Steiniger, Neustadt (DE); Steffen Mass, Bubenheim (DE); Stefan Rittinger, Mannheim (DE); Stephan Schlitter, Limburgerhof (DE); Ludwig E. Heck, Edingen-Neckarhausen (DE); Todd C. Spengeman, Missouri City, TX (US); Jeffrey T. Andress, Lake Jackson, TX (US)

Correspondence Address: OBLON, SPIVAK, MCCLELLAND MAIER & NEUSTADT, P.C., 1940 DUKE STREET, ALEXANDRIA, VA, 22314, US

(73) Assignee: BASF Aktiengesellschaft, Ludwigshafen (DE)

(21) Appl. No.: 11/476,161

(22) Filed: Jun. 28, 2006

(51) Int. Cl. C07C 29/74

(52) U.S. Cl. 568/854

(57) ABSTRACT

A process is provided for the preparation of polymethylol compounds of formula (I):

(HOCH₂)₂—C—(R)₂ , (I)

in which the radicals R independently of one another are each a further methylol group, an alkyl group having from 1 to 22 C atoms or an aryl or aralkyl group having from 6 to 22 C atoms, by
(a) condensing aldehydes having from 2 to 24 C atoms with formaldehyde in an aldol reaction using tertiary amines as a catalyst to give alkanals of formula (II):

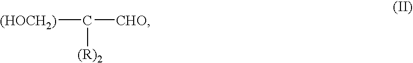

in which the radicals R independently of one another are each as defined above,
(b) then separating, by distillation, the reaction mixture obtained into a bottom product comprising predominantly the compounds of formula II and a low-boiling stream consisting of unconverted or partially converted starting materials, and
(c) hydrogenating the distillation bottom, wherein the aldol reaction is carried out with an aqueous formaldehyde solution having a methanol content of 0.35 to 0.5% by weight of methanol, the low-boiling stream is separated off at a pressure of 1 to 3 bar and temperatures of 100 to 135° C. and completely or partially recycled into the aldol reaction.

This procedure advantageously makes it possible specifically to prevent the formation of by-products and hence to increase the yield of the desired polymethylol compound.

We have found that this object is achieved by a process for the preparation of polymethylol compounds of formula (I):

(HOCH₂)₂—C—(R)₂ , (I)

in which the radicals R independently of one another are each a further methylol group, an alkyl group having from 1 to 22 C atoms or an aryl or aralkyl group having from 6 to 22 C atoms, by
(a) condensing aldehydes having from 2 to 24 C atoms with formaldehyde in an aldol reaction using tertiary amines as a catalyst to give alkanals of formula (II):

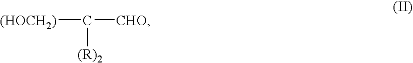

in which the radicals R independently of one another are each as defined above,
(b) then separating, by distillation, the reaction mixture obtained (aldolization product) into a bottom product comprising predominantly the compounds of formula II and a low-boiling stream consisting of unconverted or partially converted starting materials, and
(c) hydrogenating the distillation bottom, wherein the aldol reaction is carried out with an aqueous formaldehyde solution having a methanol content of 0.35 to 0.5% by weight, the separation of the low-boiling stream is effected at a pressure of 1.1 to 3 bar, preferably 1.5 bar, and a temperature of 100 to 135° C., preferably of 102 to 125° C., and the low-boiling stream is completely or partially recycled into the aldol reaction, preferably the entire low-boiling stream being recycled.

In the aldol reaction, a partially converted starting compound of formula (III):

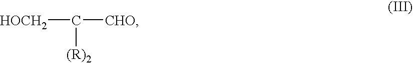

can also be formed in which the radicals R independently of one another are each hydrogen or are as defined above. According to the invention, this partially converted starting compound of formula (III), together with the desired alkanal of formula (II), is separated from the other by-products and the unreacted starting compounds and reacted again in an aldol reaction with formaldehyde having a methanol content of 0.35% by weight to 0.5% by weight, using tertiary amines as a catalyst.

The present invention relates to a process for the preparation of polymethylol compounds, which are generally also called polyalcohols, examples being neopentyl glycol or trimethylolpropane, by the hydrogenation process wherein the aldol reaction is carried out with an aqueous formaldehyde solution having a methanol content of 0.35 to 0.5% by weight of methanol, the separation of the low-boiling stream from the aldolization product is effected at a pressure of 1.1 to 2 bar and at 100 to 135° C., and the low-boiling stream is completely or partially recycled into the aldol reaction.

In industry these polyols are usually prepared by the Cannizzaro process. To prepare neopentyl glycol by this process, isobutyraldehyde is reacted with excess formaldehyde in the presence of an inorganic base, with the simultaneous formation of an inorganic formate in addition to one equivalent of the polyol as a coproduct. Separation of the salt from the neopentyl glycol is complicated and incurs additional expense. In addition, the yields in this inorganic Cannizzaro reaction, based on the isobutyraldehyde, are unsatisfactory because high-boiling components are formed during the reaction which cannot be utilized further.

Similar problems to those outlined for neopentyl glycol arise in the preparation of other triols such as trimethylolpropane (from n-butyraldehyde and formaldehyde) or trimethylolbutane (from n-pentanal and formaldehyde). To avoid these disadvantages, WO 98/28253 has disclosed a process for the preparation of polyalcohols wherein aldehydes having from 2 to 24 C atoms are first condensed with formaldehyde to the corresponding alkanals in an aldol reaction using tertiary amines as a catalyst, and then hydrogenated to the corresponding polyalcohols. The proportion of coproduct is low in this process, also called the hydrogenation process, yet methanol, in addition to the methanol introduced with the aqueous formaldehyde, is also partly formed to a small extent from the coproduct. Higher methanol contents lead to by-products such as trimethylolpropane-formaldehyde-methyl acetal and 3,3-dimethoxy-2,2-dimethyl-propanol in trimethylol production and so reduce the yield. Following aldolization, methanol is generally removed together in the course of the removal of unconverted aldehyde, since it is very difficult to separate off because of its similar boiling point. When the aldehyde thus removed comes to be used again in the aldol reaction, therefore, there is a gradual buildup in the level of methanol. In addition, there may also be increased by-product formation, depending on the polyol produced.

DE-A 199 63 438 discloses reducing the formation of these by-products and increasing the yield of the desired polymethylol compound by using for the aldol condensation an aqueous formaldehyde solution having a methanol content of <0.3% by weight. This is disadvantageous in that the low methanol content of the formaldehyde solution makes for a formaldehyde solution that is particularly prone to forming solid precipitates, since methanol stabilizes the formaldehyde solution against precipitation of solid material. The column used for removing the methanol from the aqueous formaldehyde therefore clogs rapidly in continuous operation in the base-of-column region in particular. It is consequently necessary to shut down and clean. In addition, the production of formaldehyde having an extremely low methanol content of less than 0.1% is associated with a significantly increased energy consumption.

The use of aqueous formaldehyde solution having a higher methanol concentration of 0.1 to 15% is described byWO 98/17614 in an operation for producing neopentyl glycol. The aldolization effluent is separated by means of a water-insoluble extractant, for example octanol, into an organic phase comprising the desired neopentyl glycol and an aqueous phase. The organic phase is subsequently distilled to remove, as shown for example by table 1 of WO 98/17614, low boilers such as methanol not removed by the extraction. This process is disadvantageous in that the low boilers first have to undergo a costly and inconvenient extraction step and a subsequent distillation before being recycled into the aldolization.

Proceeding from this state of the art, it is an object of the present invention to provide a process for the preparation of polymethylol compounds by condensing aldehydes with formaldehyde in an aldol reaction using tertiary amines as a catalyst to give the corresponding alkanals, and then hydrogenating the latter, as disclosed e.g. by WO 98/28253 (incorporated herein by reference) whereby a higher yield of the desired polymethylol compound is made possible through virtually complete removal of the aldehyde combined with virtually complete removal of methanol. The additional reduction obtained in the by-produced methyl ether and methanol-formaldehyde acetals enhances the resulting yield increase, depending on the polyol produced.

We have found that this object is achieved by a process for the preparation of polymethylol compounds of formula (I):

(HOCH₂)₂—C—R² (I),

in which the radicals R independently of one another are each a further methylol group, an alkyl group having from 1 to 22 C atoms or an aryl or aralkyl group having from 6 to 22 C atoms, by
(a) condensing aldehydes having from 2 to 24 C atoms with formaldehyde in an aldol reaction using tertiary amines as a catalyst to give alkanals of formula (II):

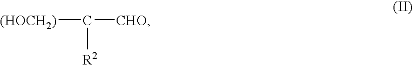

in which the radicals R independently of one another are each as defined above,
(b) then separating, by distillation, the reaction mixture obtained (aldolization product) into a bottom product comprising predominantly the compounds of formula II and a low-boiling stream consisting of unconverted or partially converted starting materials, and
(c) hydrogenating the distillation bottom, wherein the aldol reaction is carried out with an aqueous formaldehyde solution having a methanol content of 0.35 to 0.5% by weight, the separation of the low-boiling stream is effected at a pressure of 1.1 to 3 bar, preferably 1.5 bar, and a temperature of 100 to 135° C., preferably of 102 to 125° C, and the low-boiling stream is completely or partially recycled into the aldol reaction, preferably the entire low-boiling stream being recycled.

Commercially available formaldehyde is conventionally marketed in aqueous solution with concentrations of generally 30, 37, 40 and 49% by weight. Due to its manufacture by the dehydrogenation of methanol, and for its stabilization, this technical-grade formaldehyde can comprise up to 10% of methanol. By using an aqueous formaldehyde solution with a reduced methanol content according to the invention, it has been found that said by-products are formed in advantageously smaller amounts. According to the invention, an aqueous formaldehyde solution with a content of 0.4% by weight of methanol is preferably used.

On the one hand, such a formaldehyde with a reduced methanol content according to the invention can be prepared by distillative work-up in a column having about 30 to 100 plates, preferably 40 to 60 plates, at a pressure of about 0.5 to 2 bar. On the other hand, however, it is possible to use a formaldehyde or an aqueous formaldehyde solution which already has the desired low methanol content as a result of specifically adjusting the synthesis conditions in the preparation of the formaldehyde.

Although it has already been disclosed in the state of the art to use methanol-impoverished formaldehyde in the preparation of polymethylolalkanes, this disclosed state of the art applies only to trimethylolpropane obtained by the Cannizzaro reaction. Thus PL-A-162729 describes the synthesis of trimethylolpropane by reacting butyraldehyde with formaldehyde having a methanol content of <2% by weight.

Likewise, DE-A-32 07 746 and EP-A-088 275 describe the preparation of trimethylolpropane with formaldehyde having a methanol content of <0.07 mole per mole of formaldehyde in a Cannizzaro reaction.

In the aldol reaction, a partially converted starting compound of formula (III):

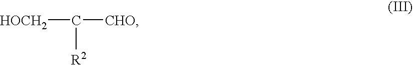

can also be formed in which the radicals R independently of one another are each hydrogen or are as defined above. According to the invention, this partially converted starting compound of formula (III), together with the desired alkanal of formula (II), is separated from the other by-products and the unreacted starting compounds and reacted again in an aldol reaction with formaldehyde having a methanol content of 0.35% by weight to 0.5% by weight, using tertiary amines as a catalyst.

In the implementation of the process according to the invention, which will be described for the preparation of neopentyl glycol by way of example, without however implying a limitation, isobutyraldehyde is first reacted in an aldol reaction with an aqueous solution of formaldehyde having a methanol content of 0.35% by weight to 0.5% by weight, and with a catalyst in the form of a tertiary amine to give hydroxypivalinaldehyde initially. The reaction mixture also comprises unconverted isobutyraldehyde, formaldehyde, and said amine catalyst and, if appropriate, water.

Said reaction mixture is then introduced into a distillation device, in which it is separated into more and less volatile components. The distillation device can consist of one or more conventional distillation columns such as columns having distillative internals such as ordered packings, random packings or trays, for example valve trays, and/or dividing wall columns having comparable internals. Here the distillation conditions are chosen so as to form a fraction of low-boiling components which comprises unreacted isobutyraldehyde, formaldehyde if appropriate, water if appropriate, and part of the amine catalyst if appropriate as the main components. This so-called low-boiling fraction is re-used in the aldol reaction, as already described. This has the advantage that unrequired isobutyraldehyde and formaldehyde and the amine catalyst can be utilized.

However, the higher yields of the desired alkanal or (after hydrogenation) of the desired polyol to be expected by carrying out the reaction in this way were not initially apparent in practice. Instead, a constantly increasing methanol concentration is observed in the aldolization in continuous operation. A constantly reduced conversion was observed due to the reduced residence time. In addition, in particular in the preparation of trimethylolpropane, the formation of by-products such as the ethers and acetals already mentioned was observed. A variety of experiments then showed that in the range, as claimed according to the invention, of methanol content of the formaldehyde used, reduces the formation of these very by-products, allowing the desired increase in the yield of alkanal or polyol.

After separation of the low-boiling fraction, the distillative work-up described leaves a less volatile bottom product consisting essentially of hydroxypivalinaldehyde in the case of neopentyl glycol preparation.

In the case of polymethylol compounds, in the preparation of which a mixture of monomethylolalkanal and dimethylolalkanal is obtained, the bottom product is again subjected to an aldol reaction by adding fresh formaldehyde having the low methanol content claimed according to the invention, and more amine catalyst if required.

In this process, the monomethylolbutanal still remaining is converted to dimethylol-butanal, and the latter is in turn obtained from the resulting reaction mixture by distillative separation of the low-boiling components such as water, formaldehyde and amine catalyst. Here again the low-boiling fraction can be recycled into the original aldol reaction, as a result of which the methylene compound formed in this secondary reaction, such as, for example, ethylacrolein, can also be converted to dimethylol-alkanal and the unconsumed formaldehyde and the amine catalyst can be utilized.

Alternatively, the bottom product, if appropriate with a further addition of amine catalyst, can be introduced into a secondary reactor in which monomethylolalkanal is converted to ethylacrolein with the elimination of water. This reaction mixture is subsequently subjected to a further distillative separation in order to separate off water, formaldehyde, ethylacrolein and amine catalyst and recycle these components into the aldol reaction.

Due to the inventively methanol content of the aqueous formaldehyde solution, there is no risk of a substantial formation of ether and acetal by-products. However, without reducing the methanol content, this by-product formation would have an extremely unfavorable effect on the yield of the desired end product in the reaction procedure represented here, where both the ethylacrolein, formed, for example, in the trimethylol-propane preparation, and the formaldehyde are substantially totally converted or utilized.

The aldol reaction is generally carried out at a temperature of 5 to 100° C., preferably 15 to 80° C., and the residence time is generally adjusted to 0.25 to 12 hours according to the temperature.

In the aldol reaction used for the preparation of neopentyl glycol, the molar ratio of freshly added isobutyraldehyde to the amount of formaldehyde introduced is appropriately between 1:2 and 1:5, preferably 1:2 to 1:3.5. The amount of tertiary amine catalyst added in the aldol reaction is normally 0.001 to 0.2, preferably 0.01 to 0.07 equivalent, based on the isobutyraldehyde added, i.e. the amine is used in catalytic amounts.

The subsequent distillation for separation into a low-boiling fraction and the bottom product is generally carried out at 100 to 135° C., preferably 103 to 125° C., and a pressure of 1.1 to 2 bar, preferably 1.5 bar. The low-boiling fraction is recycled at the pressure prevailing in the distillation. Preferably, the entire low-boiling fraction obtained is recycled into the aldolization. After the aldol reaction and the low-boiling component separation have been carried out, in the preparation of neopentyl glycol, an aldolization product consisting essentially of hydroxypivalinaldehyde is obtained, which is catalytically hydrogenated.

If, after the aldol reaction, in particular for the preparation of trimethylolpropane, and the subsequent distillative separation of the low-boiling components, a bottom product is obtained which consists essentially of dimethylolalkanal and monomethylolalkanal, the latter reacting substantially totally in another secondary aldol reaction to give dimethylolbutanal and being isolated again in a distillative separation, an aldolization product is obtained which consists essentially of dimethylolbutanal or, according to the starting compounds used, the corresponding alkanal.

The respective aldolization product is catalytically hydrogenated in a hydrogenation reactor. Thus the hydroxypivalinaldehyde obtained in the neopentyl glycol preparation according to the invention or the dimethylolalkanal in the preparation of trimethylol-propane according to the invention is catalytically hydrogenated in a conventional manner with hydrogen to form neopentyl glycol or for example in the manner described in WO 98/28253 to form trimethylolpropane.

Suitable hydrogenation catalysts are especially copper-containing supported catalysts such as those described in WO-A-95/32171. Other suitable catalysts are those described in EP-A-044 444, EP-A-044 412 or DE-A-19 57 592. The hydrogenation is appropriately carried out continuously, e.g. in a reactor tube packed with catalyst, in which the reaction solution is passed over the catalyst bed, e.g. by the trickle method or in the region of the transition flow, as described in DE-A-19 41 633 or DE-A-20 40 501. It can be advantageous to recycle part of the reaction discharge stream, if appropriate with cooling, and pass it over the fixed catalyst bed again. Similarly, it can be advantageous to carry out the hydrogenation in several reactors connected in series, for example 2 to 4 reactors, the hydrogenation reaction proceeding only to a partial conversion of e.g. 50 to 98% in the individual reactors upstream of the last reactor, and the hydrogenation only being brought to completion in the last reactor. It can be appropriate here to cool the hydrogenation discharge from one reactor before it enters the next reactor, for example by means of cooling devices, by injecting cold gases such as hydrogen or nitrogen, or by introducing part of the cold reaction solution stream.

The hydrogenation temperature is generally between 50 and 180° C., preferably between 90 and 140° C. The applied hydrogenation pressure is generally 10 to 250 bar, preferably 20 to 120 bar.

The hydrogenation can be carried out with the addition of an inert solvent. Solvents which can be used are both cyclic ethers, such as THF or dioxane, and acyclic ethers, as well as lower alcohols, e.g. methanol, ethanol or 2-ethylhexanol.

It is also possible to use any desired hydrogenation methods and hydrogenation catalysts, such as those conventionally used for the hydrogenation of aldehydes and described in detail in the standard literature.

The resulting crude neopentyl glycol or crude trimethylolpropane can be purified by distillation in a manner conventional per se.

The process according to the invention can be carried out with or without the addition of organic solvents or solubilizers. The addition of solvents or solubilizers can prove particularly advantageous when using long-chain aldehydes as starting materials. The use of solvents which form suitable low-boiling azeotropic mixtures with the low-boiling compounds in the individual distillations of the process according to the invention may reduce the energy expenditure in these distillations and/or facilitate the distillative separation of the low-boiling components from the high-boiling compounds.

Examples of suitable solvents are cyclic and acyclic ethers, such as THF, dioxane and methyl tert-butyl ether, or alcohols, such as methanol, ethanol or 2-ethylhexanol.

The reaction procedures described for the aldolization reaction can be carried out at a pressure generally of 1 to 30 bar, preferably 1 to 15 bar and particularly preferably 1 to 5 bar, appropriately under the autogenous pressure of the reaction system in question.

The novel process is applicable to practically any alkanals with a methylene group in the α-position to the carbonyl group. Starting materials which can be used are aliphatic aldehydes having from 2 to 24 C atoms which can be linear or branched or else can comprise alicyclic groups. It is also possible to use araliphatic aldehydes as starting materials, provided that they comprise a methylene group in the α-position to the carbonyl group. Aralkylaldehydes having from 8 to 24 C atoms, preferably from 8 to 12 C atoms, are generally used as starting materials, an example being phenyl-acetaldehyde. It is preferable to use aliphatic aldehydes having from 2 to 12 C atoms, for example 3-ethyl-, 3-n-propyl-, 3-isopropyl-, 3-n-butyl-, 3-isobutyl-, 3-sec-butyl- and 3-tert-butyl-butanal and the corresponding n-pentanals, n-hexanals and n-heptanals; 4-ethyl-, 4-n-propyl-, 4-isopropyl-, 4-n-butyl-, 4-isobutyl-, 4-sec-butyl- and 4-tert-butyl-pentanals, -n-hexanals and -n-heptanals; 5-ethyl-, 5-n-propyl-, 5-isopropyl-, 5-n-butyl-, 5-isobutyl-, 5-sec-butyl- and 5-tert-butyl-n-hexanals and -n-heptanals; 3-methylhexanal and 3-methylheptanal; 4-methylpentanal, 4-methylheptanal, 5-methylhexanal and 5-methylheptanal; 3,3,5-trimethyl-n-pentyl-, 3,3-diethylpentyl-, 4,4-diethylpentyl-, 3,3-dimethyl-n-butyl-, 3,3-dimethyl-n-pentyl-, 5,5-dimethylheptyl-, 3,3-dimethylheptyl-, 3,3,4-trimethylpentyl-, 3,4-dimethylheptyl-, 3,5-dimethylheptyl-, 4,4-dimethylheptyl-, 3,3-diethylhexyl-, 4,4-dimethylhexyl-, 4,5-dimethylhexyl-, 3,4-dimethylhexyl-, 3,5-dimethylhexyl-, 3,3-dimethylhexyl-, 3,4-diethylhexyl-, 3-methyl-4-ethylpentyl-, 3-methyl-4-ethylhexyl-, 3,3,4-trimethylpentyl-, 3,4,4-trimethylpentyl-, 3,3,4-trimethyl-hexyl-, 3,4,4-trimethylhexyl- and 3,3,4,4-tetramethylpentyl-aldehyde; C₂ to C₁₂ n-alkanals are used in particular.

It is also possible to prepare e.g. 2-ethylacrolein, 2-methylacrolein or 2-hydroxymethyl-acrolein from 2-alkylacroleins or acrolein by reaction with water and formaldehyde having a reduced methanol content according to the invention. Apart from the already mentioned neopentyl glycol and trimethylolpropane, which have been referred to here as the main examples, acetaldehyde, propionaldehyde and n-pentanal can also preferably be used as starting compounds for the preparation of pentaerythritol, trimethylolethane and trimethylolbutane respectively.

Possible tertiary amines are those known per se for their suitability for the condensation of aldehydes with formaldehyde, examples being those described in DE-A-28 13 201 and DE-A-27 02 582. Particularly preferred tertiary amines are tri-n-alkyl-amines such as triethylamine, tri-n-propylamine, tri-n-butylamine and especially trimethylamine.

The process according to the invention is distinguished by high yields, based both on the starting aldehyde and on the formaldehyde, and gives rise to very low losses of amine catalyst. Because the process operates at relatively low pH values, no Cannizzaro reaction takes place, so the formation of formate salts as coupling products is avoided.

The invention will be illustrated in greater detail below with the aid of Examples.

### EXAMPLE 1

Formaldehyde

A 49% by weight aqueous formaldehyde solution with a methanol content of 2.0% by weight was distilled at a top pressure of 1.2 bar in a bubble-cap column comprising 45 trays. The feed rate at the 30th tray was 4 kg/h. A product containing 69.4% of methanol and 12% of formaldehyde was withdrawn from the top at a rate of about 75 g/h. 4 kg/h of steam were fed to the heating surfaces in the bottom evaporator. A residual methanol concentration in the bottom discharge of 0.4% by weight was established. The formaldehyde distilled in this way is used for the aldolization reaction described below:

Aldol Reaction

The aldol reaction was carried out in a stirred tank cascade consisting of two heatable stirred tanks interconnected by overflow tubes. The stirred tanks were charged continuously with 700 g/h of a fresh 49% aqueous formaldehyde solution freed of methanol as described above, and 750 g/h of isobutyraldehyde and 40 g/h of fresh trimethylamine as catalyst in the form of a 50% aqueous solution. The temperature in both stirred tanks was about 70° C. With the exception of the reduction in methanol content for the purposes of the present reaction, the reaction procedure is the same as that already indicated in Example 8 of WO 98/28253, the content of whose disclosure is expressly incorporated herein by way of reference.

The effluent was passed directly into the upper region of a column equipped with 1.5 m fabric packing (500 m²/m³ specific surface area) in the rectifying section and 4 m sheet metal packing (250 m²/m³), above the sheet metal packing, where it was separated by distillation at a pressure of 1.5 bar and a bottom temperature of 102° C. into a low-boiling top product, essentially comprising isobutyraldehyde, formaldehyde, water and trimethylamine, and a high-boiling bottom product. The top product was continuously condensed and recycled into the reactors described above at the same pressure.

The bottom product was found to have an isobutyraldehyde concentration of about 0.1%. A methanol concentration of about 1% was achieved into the recycled low boiler (50 g/h).

Hydrogenation

a) Hydrogenation catalyst
A 20% by weight sodium carbonate solution and an aqueous solution I comprising 2.67% by weight of Al and 5% by weight of Cu in the form of their nitrates were used as feedstocks.
In the precipitation stage, solution I and sodium carbonate solution were metered at 80° C. into a precipitating tank such that a pH of 5.6 became established.
The precipitation mix was transferred into a larger stirred vessel and adjusted therein at 80° C. to a pH of 7.9 with sodium carbonate solution. The suspension was then routed onto a filter press.
The mix was then filtered and washed nitrate free with water. The filter paste was suspended in water and dried in a spray tower with hot air at 130-150° C. exit temperature. This was followed by a calcination at a temperature of 375-390° C. The powder was subsequently tableted with 3% by weight of graphite as adjuvant to form 3×3 mm tablets. The tablets obtained were then calcined in a heated rotary tube at 600° C. for 60 min.
This catalyst consisted of 55% by weight of CuO and 45% by weight of Al₂O₃ and had a specific surface area of 85 m²/g, an Hg porosity of 0.38 ml/g coupled with a bulk density of 1042 g/l.

b) In addition to water, the bottom product obtained above comprised essentially hydroxypivalinaldehyde, formaldehyde, trimethylammonium formate and traces of isobutyraldehyde. This bottom product was then subjected to continuous hydrogenation. This was done by hydrogenating the reaction solution over the hydrogenation catalyst described above at 40 bar and 115° C. in a primary reactor by the loop/trickle method and in a downstream secondary reactor by the loop method. The apparatus used consisted of a heated primary reactor with a length of 10 m (internal diameter: 27 mm). The loop throughput was 25 l/h of liquid and the reactor feed was adjusted to 4 kg/h, corresponding to a hydrogenation discharge of 4 kg/h. The yield of neopentyl glycol after hydrogenation was 97%.

### COMPARATIVE EXAMPLE 2

The procedure in this comparative example was exactly the same as that indicated in example 1 except that the formaldehyde used comprised 2.0% by weight of methanol instead of 0.4% and the low-boiling fraction was removed at a pressure of 1 bar. In the bottom product of the distillation a concentration of approximately 0.7% iso-butyraldehyde was analyzed and in the low boilers removed overhead and recycled (100 g/h) a methanol concentration of approximately 10% was analyzed. The yield was reduced to 94% neopentyl glycol based on isobutyraldehyde.

1. A process for the preparation of polymethylol compounds of formula (I):
(HOCH₂)₂—C—(R)₂ (I),
in which the radicals R independently of one another are each a further methylol group, an alkyl group having from 1 to 22 C atoms or an aryl or aralkyl group having from 6 to 22 C atoms, by
(a) condensing aldehydes having from 2 to 24 C atoms with formaldehyde in an aldol reaction using tertiary amines as a catalyst to give alkanals of formula (II): 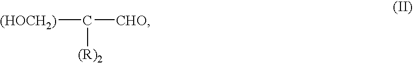
in which the radicals R independently of one another are each as defined above,
(b) then separating, by distillation, the reaction mixture obtained into a bottom product comprising predominantly the compounds of formula II and a low-boiling stream consisting of unconverted or partially converted starting materials, and
(c) hydrogenating the distillation bottom, wherein the aldol reaction is carried out with an aqueous formaldehyde solution having a methanol content of 0.35 to 0.5% by weight of methanol, the low-boiling stream is separated off at a pressure of 1 to 3 bar and temperatures of 100 to 135° C. and recycled into the aldol reaction.

2. The process according to claim 1 wherein the low-boiling stream is separated off at 1.5 bar.

3. The process according to claim 1 wherein the aldol reaction is carried out with an aqueous formaldehyde solution having a methanol content of 0.4% by weight.

4. The process according to claim 1 wherein the reaction is carried out continuously.

5. The process according to claim 1 wherein the tertiary amine catalyst is used in an amount such that the pH of the reaction mixture is 5 to 12.

6. The process according to claim 1 wherein trimethylamine is used as the catalyst.

7. The process according to claim 1 wherein at least one of propionaldehyde, n-butyraldehyde, acetaldehyde or isobutyraldehyde is converted to at least one of trimethylolethane, trimethylol propane, pentaerythritol or neopentyl glycol respectively.

8. The process according to claim 1 wherein said aldehydes having from 2 to 24 C atoms is at least one aldehyde selected from the group consisting of propionaldehyde, n-butyraldehyde, acetaldehyde or isobutyraldehyde.

9. The process according to claim 1, wherein said aqueous formaldehyde solution has a methanol content of from 0.35 to 0.4 % by weight.

10. The process according to claim 1, wherein said aldol reaction is conducted at a temperature of 15 to 80°C.

11. The process according to claim 1, wherein said aldol reaction is conducted with a residence time of 0.25 to 12 hours.

12. The process according to claim 1, wherein said alkanal of formula (II) is neopentyl glycol formed at a molar ratio of isobutyraldehyde to formaldehyde of 1:2 to 1:5.

13. The process according to claim 12, wherein said tertiary amine is used as a catalyst in an amount of 0.001 to 0.2 equivalents based on said isobutyraldehyde.
